(19) United States
(12) Patent Application Publication
(10) Pub. No.: US 2018/0310838 A1
(43) Pub. Date: Nov. 1, 2018

(54) Pressing Method and Pressing Apparatus For Measuring Pressure Pulse Wave

(71) Applicant: Korea Institute Of Oriental Medicine, Yuseong-gu Daejeon (KR)

(72) Inventors: Young Ju Jeon, Yuseong-gu Daejeon (KR); Jang Han Bae, Yuseong-gu Daejeon (KR); Jae Uk Kim, Yuseong-gu Daejeon (KR); Young Min Kim, Yuseong-gu Daejeon (KR); Jong Yeol Kim, Jung-gu Daejeon (KR)

(21) Appl. No.: 15/528,028

(22) PCT Filed: Dec. 5, 2014
(86) PCT No.: PCT/KR2014/011865

§ 371 Date: May 18, 2017

(30) Foreign Application Priority Data
Nov. 25, 2014 (KR) 10-2014-0165171

(51) Int. Cl. A61B 5/021; A61B 5/02

(52) U.S. Cl. CPC A61B 5/02116; A61B 5/022; A61B 5/02007

(57) ABSTRACT

Disclosed are a pressing method and a pressing apparatus for measuring pressure pulse waves by continuously pressing, with a constant velocity, an object such as the human body by using a pressing tool, and then identifying a point when the amplitude of the pressure pulse wave being measured changes from increasing to decreasing, to accurately recognize a pressure reducing control point for the pressing tool so that the amplitude of the pressure pulse wave is retained at an appropriate level while measuring. The pressing method comprises the steps of: measuring a pressure pulse wave from an object; determining a pressure reducing point when the amplitude of the pressure pulse wave that is measured changes from increasing to decreasing, while pressure is applied to the object by the pressing tool; and controlling the pressing tool so that pressure applied to the object is decreased after the pressure decreasing point.

### TECHNICAL FIELD

Example embodiments relate to a pressure applying method and apparatus for measuring a pressure pulse wave by continuously applying a pressure to an object, for example, a human body, at a constant speed using a pressurizer, and determining a contact pressure reduction point at which an amplitude of a pressure pulse wave being measured changes from increasing to decreasing, thereby recognizing an accurate contact pressure reduction control point for the pressurizer to reduce the contact pressure and maintaining a desirable amplitude of the pressure pulse wave to be measured.

### BACKGROUND ART

A doctor uses a pulse-feeling method, as an existing pulse-based diagnosing method, to diagnose an illness or disease by feeling a pulse of a patient from a certain point of an artery, for example, a Chon portion, a Gwan portion, and a Cheok portion, and observing a change in the pulse of the patient.

Such a pulse-feeling method is used to determine a presence or absence of a functional abnormality of an organ by determining a balance between energy and blood flowing in a human body, and determine a health condition by detecting a strength of a pulse, or a strength of a pressure, and a cycle of the pulse while three fingers of the doctor are being in contact with three points of the Chon portion, the Gwan portion, and the Cheok portion. A pressure applying or pressurizing method used by an oriental medical doctor when feeling a pulse of a patient may be broadly classified into two methods—one is to gradually apply a pressure to a point of the patient from which a pulse is to be measured and the other is to detect a pulse pattern by fully applying a pressure to such an extent that a pulse is no longer felt and then gradually reducing the applied pressure, or contact pressure as used herein.

However, the pulse-feeling method may require a great amount of time from a doctor to be skilled in using such a method and a great deal of experience from a doctor to diagnose a disease and treat a patient accordingly. In the method, a condition of a patient and a severity of a disease may be determined depending on a sense of fingers of a doctor, and thus standardizing or systematizing various states or conditions based on a constitution of a patient may not be easy. In addition, there may be a risk of a misdiagnosis.

To overcome such challenges described in the foregoing, there is provided a method of electrically applying a pressure to a target portion of a human body from which a pulse is to be measured using a pressurizer, collecting an output pulse waveform of the human body, and diagnosing a health condition of the human body.

FIG. 1 is a diagram illustrating examples of an existing measurement method of collecting pressure pulse waves according to a related art. An (a) portion of FIG. 1 is a diagram illustrating an example of a method of collecting pressure pulse waves by applying a pressure at a constant speed, and a (b) portion of FIG. 1 is a diagram illustrating an example of a method of collecting pressure pulse waves by gradually applying a pressure in a stepwise manner.

Referring to the (a) portion of FIG. 1, in an example of the existing measurement method, a pressure pulse wave may be measured by continuously applying a pressure to an object, for example, an aorta radialis of a human body, at a constant speed using a pressurizer.

Referring to the (b) portion of FIG. 1, in another example of the existing measurement method, a pressure pulse wave may be measured by gradually increasing a pressure to be applied to an object, for example, an aorta radialis of a human body, using a pressurizer.

A magnitude, or amplitude, of a pressure pulse wave being measured may gradually increase at an initial stage, and then decrease after a maximum amplitude of the pressure pulse wave is measured.

In such an existing measurement method, a point at which an amplitude of a pressure pulse wave starts decreasing after increasing may be recognized as a limit point in measurement, and a measurer may manually terminate the measurement.

However, in the existing measurement method, the measurement may be immediately terminated due to such a decrease in the amplitude of the pressure pulse wave being measured, and thus determining a change pattern of the pressure pulse wave at a point at which the maximum amplitude of the pressure pulse wave is output may not be easy.

In addition, in the existing measurement method, a measurement range of the maximum amplitude of the pressure pulse wave may be extremely short, and thus obtaining a sufficiently high-frequency resolution may not be easy.

Thus, there is a desire for a new model that may enable a high-frequency resolution, compared to the existing measurement method, in a frequency component analysis of a pressure pulse wave, and determine an accurate change pattern of a pressure pulse wave at a point at which a maximum amplitude of the pressure pulse wave is output.

### DISCLOSURE

### Technical Goals

An aspect of the present disclosure provides a pressure applying method and apparatus for measuring a pressure pulse wave, which may recognize, as a contact pressure reduction control point for a pressing tool, or a pressurizer, to reduce a contact pressure, a point at which an amplitude of a pressure pulse wave decreases after increasing by continuously applying a pressure at a constant speed, and may determine a change pattern of the pressure pulse wave after a maximum amplitude of the pressure pulse wave is measured.

In addition, the pressure applying method and apparatus may control the pressurizer to maintain the contact pressure based on a change in an amplitude of the pressure pulse wave after controlling the pressurizer to reduce the contact pressure, and expand a measurement range to obtain a relatively high-frequency resolution in a frequency component analysis of the pressure pulse wave.

Another aspect of the present disclosure provides a pressure applying method and apparatus for measuring a pressure pulse wave, which may flexibly control a pressurizer to apply a pressure, reduce a contact pressure, or maintain a contact pressure based on an amplitude of a pressure pulse wave being measured, and thus obtain a wide range of feature values associated with the pressure pulse wave.

### Technical Solutions

According to an aspect of the present disclosure, there is provided a pressure applying method including measuring a pressure pulse wave from an object, determining a contact pressure reduction point at which an amplitude of the measured pressure pulse wave is reversed from increasing to decreasing by applying a pressure to the object through a pressurizer, and controlling the pressurizer to reduce the pressure applied to the object after the contact pressure reduction point.

According to another aspect of the present disclosure, there is provided a pressure applying method including measuring a pressure pulse wave from an object by pressing the object by a pressurizer, determining a point in time at which an amplitude of the measured pressure pulse wave changes from increasing to decreasing or from decreasing to increasing, and controlling the pressurizer to reduce or maintain a pressure applied to press the object after the determined point.

According to still another aspect of the present disclosure, there is provided a pressure applying method including measuring a pressure pulse wave from an object, determining a second point at which an amplitude of the pressure pulse wave is reversed from increasing to decreasing by applying a pressure to the object until a first point at which the pressure pulse wave is not measured and reducing the pressure after the first point, and controlling the pressurizer to apply a pressure to the object after the second point.

According to yet another aspect of the present disclosure, there is provided a pressure applying apparatus including a sensor configured to measure a pressure pulse wave from an object, a processor configured to determine a contact pressure reduction point at which an amplitude of the pressure pulse wave measured by applying a pressure to the object through a pressurizer is reversed from increasing to decreasing, and a controller configured to control the pressurizer to reduce the pressure applied to the object after the contact pressure reduction point.

According to further another aspect of the present disclosure, there is provided a pressure applying apparatus configured to measure a pressure pulse wave from an object by pressing the object by a pressurizer, determine a point in time at which an amplitude of the measured pressure pulse wave changes from increasing to decreasing or from decreasing to increasing, and control the pressurizer to reduce or maintain a pressure applied to press the object after the determined point.

According to still another aspect of the present disclosure, there is provided a pressure applying apparatus configured to measure a pressure pulse wave from an object, determine a second point in time at which an amplitude of the pressure pulse wave is reversed from increasing to decreasing by applying a pressure to the object until a first point in time at which the pressure pulse wave is not measured and reducing the pressure after the first point, and control the pressurizer to apply the pressure to the object after the second point.

### Advantageous Effects

According to example embodiments of the present disclosure, a pressure applying method and apparatus for measuring a pressure pulse wave may recognize, as a contact pressure reduction control point for a pressing tool, or a pressurizer, to reduce a contact pressure, a point at which an amplitude of a pressure pulse wave decreases after increasing by continuously applying a pressure at a constant speed, and may determine a change pattern of the pressure pulse wave after a maximum amplitude of the pressure pulse wave is measured.

According to example embodiments of the present disclosure, a pressure applying method and apparatus may control a pressurizer to maintain a contact pressure based on a change in an amplitude of a pressure pulse wave after controlling the pressurizer to reduce a contact pressure, and expand a measurement range to obtain a relatively high-frequency resolution in a frequency component analysis of the pressure pulse wave.

According to example embodiments of the present disclosure, a pressure applying method and apparatus for measuring a pressure pulse wave may flexibly control a pressurizer to apply a pressure, reduce a contact pressure, or maintain a contact pressure based on an amplitude of a pressure pulse wave being measured, and thus obtain a wide range of feature values associated with the pressure pulse wave.

### BRIEF DESCRIPTION OF DRAWINGS

FIG. 1 is a diagram illustrating examples of an existing measurement method of collecting pressure pulse waves according to a related art.

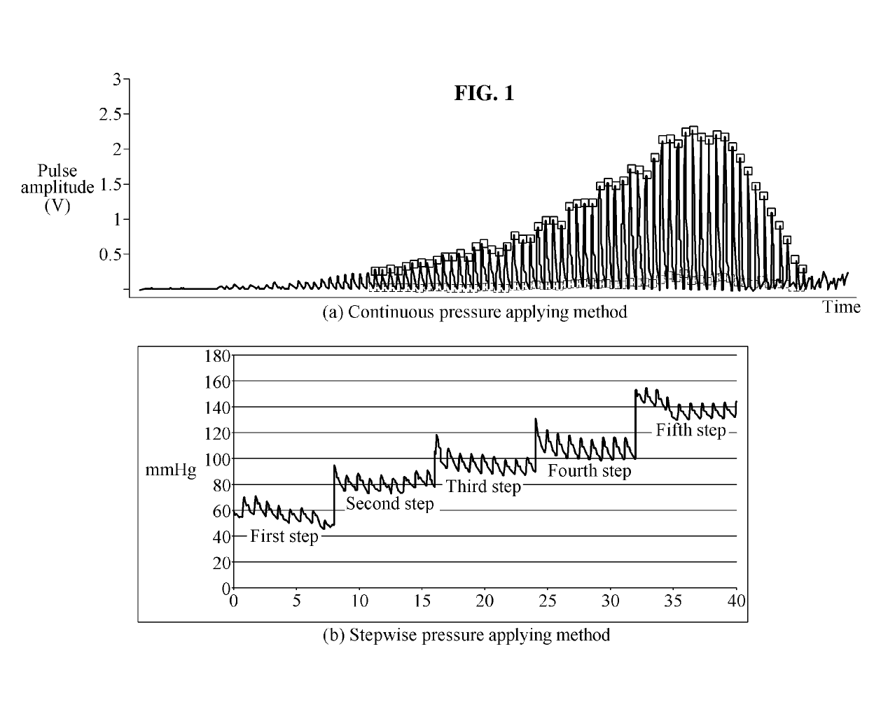

FIG. 2 is a diagram illustrating a configuration of a pressure applying apparatus according to an example embodiment.

FIG. 3 is a diagram illustrating an example of an amplitude of a pressure pulse wave to be measured by a pressure applying apparatus according to an example embodiment.

FIG. 4 is a diagram illustrating examples of a method of controlling a contact pressure by a pressure applying apparatus according to an example embodiment.

FIG. 5 is a flowchart illustrating a method of controlling a pressurizer by determining a contact pressure reduction point and a contact pressure retention point according to an example embodiment.

FIG. 6 is a flowchart illustrating a pressure applying method according to an example embodiment.

### BEST MODE FOR CARRYING OUT THE INVENTION

Hereinafter, example embodiments will be described in detail with reference to the accompanying drawings. It should be understood, however, that there is no intent to limit this disclosure to the particular example embodiments disclosed. Regarding the reference numerals assigned to the elements in the drawings, it should be noted that the same elements will be designated by the same reference numerals, wherever possible, even though they are shown in different drawings.

“A pressure pulse wave” used herein refers to a recorded time-based change in an internal blood pressure based on a transmission of a pulse wave, and also to a recorded change in an arterial pressure involved with a heartbeat. To record such a pressure pulse wave, a traditional method may include directly recording a blood pressure by intubating an artery and analyzing a change of the blood pressure at each heartbeat. However, a method of applying a pressure to skin above an artery using a pressing tool or a pressurizer, for example, a motor, and electrically monitoring a change in an arterial blood pressure may be used recently.

According to example embodiments, there is provided a pressure applying method and apparatus to measure a pressure pulse wave. The pressure applying method may include continuously applying a pressure to an object, for example, a human body, using a pressing tool or a pressurizer at a constant speed, determining a point in time at which an amplitude of a measured pressure pulse wave is reversed from increasing to decreasing, accurately recognizing a contact pressure reduction control point for the pressurizer to reduce a contact pressure, and maintaining a desirable amplitude of the pressure pulse wave to be measured.

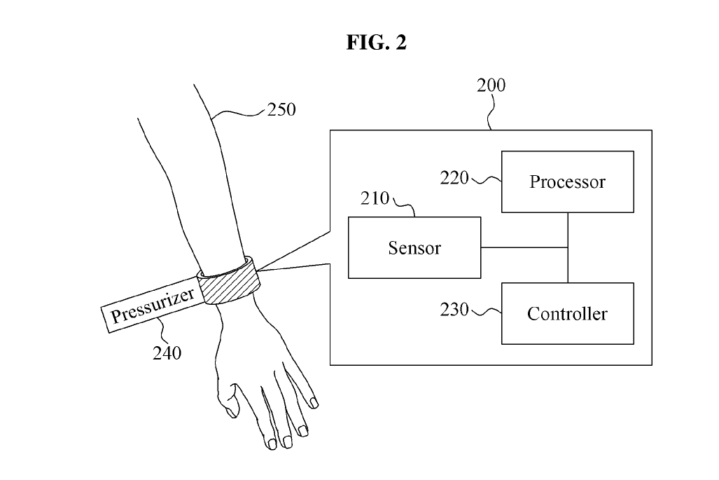

FIG. 2 is a diagram illustrating a configuration of a pressure applying apparatus according to an example embodiment.

A pressure applying apparatus 200 includes a sensor 210, a processor 220, and a controller 230. The pressure applying apparatus 200 may be attached to an object 250, which is a target from which a pressure pulse wave is to be measured.

The sensor 210 may measure or sense a pressure pulse wave from the object 250. That is, the sensor 210 may measure an amplitude of the pressure pulse wave based on a pulse wave that is generated when a blood vessel of the object 250 is gradually constricted by a pressure applied by a pressurizer 240. The amplitude of the pressure pulse wave to be measured by the sensor 210 may be measured by a unit of volt (v).

In association with the applying of the pressure, the sensor 210 may calculate an optimal contact pressure (OCP). The OCT refers to a contact pressure at a point in time at which the pressure pulse wave has a maximum magnitude or amplitude.

In calculating the OCP, the sensor 210 may calculate a difference between a maximum measured value and a minimum measured value of an amplitude of the pressure pulse wave in each preset time interval while the pressurizer 240 is applying a pressure to the object 250. Subsequently, the sensor 210 may calculate, as the OCP, an average contact pressure in a time interval having a maximum value among the calculated differences. The average contact pressure refers to an average pressure during a certain period of time.

For example, the sensor 210 may calculate a difference Δd between the maximum measured value and the minimum measured value of the amplitude of the pressure pulse wave measured at an interval of 2 seconds while the pressurizer 240 is being controlled to apply a pressure, and calculate an average value of contact pressures in a corresponding time interval at an interval of 2 seconds. Subsequently, the sensor 210 may recognize a time interval Iₘₐₓ in which a maximum difference Δd is calculated among differences Δd calculated until a current time, and determine an average pressure calculated in the recognized time interval Iₘₐₓ to be the OCP.

The sensor 210 may calculate the OCP so that the processor 220 may determine a contact pressure retention point to maintain a contact pressure without applying a pressure or reducing a contact pressure by the pressurizer 240.

The processor 220 may determine a contact pressure reduction point at which an amplitude of the pressure pulse wave measured by applying a pressure to the object 250 through the pressurizer 240 is reversed from increasing to decreasing. That is, the processor 220 may determine a point in time at which the amplitude of the pressure pulse wave is reversed to decreasing from the maximum measured value of the amplitude of the pressure pulse wave to be a contact pressure reduction control point at which the pressurizer 240 is to be controlled to reduce a contact pressure after being controlled to apply a pressure.

To determine the contact pressure reduction point, the processor 220 may numerically determine the contact pressure reduction point using the OCP and the difference Δd between the maximum measured value and the minimum measured value of the amplitude of the pressure pulse wave that is calculated by the sensor 210.

In one example, the processor 220 may determine the contact pressure reduction point when consecutively decreasing differences are calculated (example 1).

For example, under the assumption that, based on a current point, a difference between a maximum measured value and a minimum measured value of a pressure pulse wave in a previous interval between 6 second and 4 second is Δdₙ₋₃, a difference in a previous interval between 4 second and 2 second is Δdₙ₋₂, and a difference in a previous interval between 2 second and 0 second is Δdₙ₋₁, and the three differences Δdₙ₋₃, Δdₙ₋₂, and Δdₙ₋₁ calculated during 6 seconds decrease consecutively, the processor 220 may determine the contact pressure reduction point by determining a point at which the amplitude of the pressure pulse wave is reversed to decreasing from the maximum measured value.

For another example, the processor 220 may determine the contact pressure reduction point by determining that the differences decrease consecutively when the difference Δdₙ₋₁ is not greater than the difference Δdₙ₋₂ by 0.15 v or higher.

In another example, the processor 220 may determine the contact pressure reduction point when, among such consecutive differences, a lastly calculated second difference is less than an initially calculated first difference, and a difference between the first difference and the second difference is greater than a preset first threshold value (example 2).

For example, among the three differences Δdₙ₋₃, Δdₙ₋₂, and Δdₙ₋₁, the initially calculated first difference is the difference Δdₙ₋₃ and the lastly calculated second difference is the difference Δdₙ₋₁. In such an example, the processor 220 may determine the contact pressure reduction point by comparing the first difference Δdₙ₋₃ and the second difference Δdₙ₋₁, and determining that the amplitude of the pressure pulse wave is reversed from the maximum measured value when the second difference Δdₙ₋₁ becomes less than the first difference Δdₙ₋₃ and also the difference between the first difference Δdₙ₋₃ and the second difference Δdₙ₋₁, for example, Δe(Δdₙ₋₃−Δdₙ₋₁), is greater than the first threshold value, for example, 0.4 v.

Here, in a case that one difference Δd among differences continuously monitored is greater than or equal to 2.5 v, the processor 220 may adjust the first threshold value to be 0.5 v.

Example 2 described in the foregoing may be implemented independently, or implemented depending on a condition that example 1 is satisfied.

In still another example, the processor 220 may determine the contact pressure reduction point when, among the consecutive differences, a difference between two adjacent calculated differences is greater than a preset second threshold value (example 3).

For example, in a case that, among the three differences Δdₙ₋₃, Δdₙ₋₂, and Δdₙ₋₁, a difference Ae between two adjacent calculated differences Δdₙ₋₂ and Δdₙ₋₁ exceeds the second threshold value, for example, 1.2 v, the processor 220 may determine the contact pressure reduction point by determining that the amplitude of the pressure pulse wave is reversed from the maximum measured value.

That is, in example 3 described in the foregoing, the processor 220 may determine the contact pressure reduction point by verifying whether the amplitude of the pressure pulse wave decreases by a suddenly blocked blood vessel or suddenly increases by the pressurizer 240 being pressed hard against a bone.

Example 3 described in the foregoing may be implemented independently, or implemented depending on a condition that both examples 1 and 2 are not satisfied.

In yet another example, the processor 220 may determine the contact pressure reduction point when a difference above a preset threshold range is calculated and then a difference below the threshold range is calculated (example 4).

For example, under the assumption that the threshold range is 1.8 v to 1.5 v and, among the three differences Δdₙ₋₃, Δdₙ₋₂, and Δdₙ₋₁, the difference Δdₙ₋₃ is calculated to be greater than an uppermost value 1.8 v of the threshold range and then one of the differences Δdₙ₋₂ and Δdₙ₋₁ is calculated to be less than a lowermost value 1.5 v of the threshold range, the processor 220 may determine the contact pressure reduction point by determining that the amplitude of the pressure pulse wave is reversed from the maximum measured value.

In example 4 described in the foregoing, the processor 220 may determine the contact pressure reduction point by determining that a pulse is sufficiently generated when a difference Δd is above the threshold range, and that another difference Δd is out of the threshold range through continuous monitoring and a blood vessel is thus slowly closed.

Example 4 described in the foregoing may be implemented independently, or implemented depending on a condition that examples 1 through 3 are not satisfied.

After the contact pressure reduction point, the controller 230 may control the pressurizer 240 to reduce the pressure applied to the object 250. That is, in response to the determination of the contact pressure reduction point, the controller 230 may change a control method from a pressure applying control to a contact pressure reduction control to control the pressurizer 240 that is previously controlled to apply a pressure to reduce the contact pressure.

Thus, by increasing again the amplitude of the pressure pulse wave reversed from the maximum measured value to decreasing, and measuring the pressure pulse wave, a change pattern of the pressure pulse wave after a maximum amplitude of the pressure pulse wave is measured may be continuously determined.

In addition, the pressure applying apparatus 200 may determine a contact pressure retention point to maintain, to be constant, the amplitude of the pressure pulse wave that is reversed again to increasing through the contact pressure reduction control, and thus expand a measurement range.

When the control method is changed to the contact pressure reduction control to control the pressurizer 240 to reduce the contact pressure, the pressurizer 240 may reduce a pressure to be applied to the object 250. Due to such a contact pressure reduction control, the amplitude of the pressure pulse wave may be reversed again from decreasing to increasing.

The processor 220 may continuously monitor a contact pressure from the pressurizer 240 that is being controlled to reduce the contact pressure, and determine the contact pressure retention point at which the monitored contact pressure becomes less than the OCP. That is, the processor 220 may determine the contact pressure retention point using the OCP that is calculated while the pressurizer 240 is being controlled to apply the pressure and the contact pressure that is measured while the pressurizer 240 is being controlled to reduce the contact pressure.

For example, after the contact pressure reduction control to control the pressurizer 240 to reduce the contact pressure, the processor 220 may determine, to be the contact pressure retention point, a point in time at which a gradually reduced contact pressure becomes less than the OCP by monitoring a contact pressure from the pressurizer 240.

Subsequently, the controller 230 may control the pressurizer 240 to maintain a contact pressure measured at the contact pressure retention point until after the contact pressure retention point. That is, in response to the determination of the contact pressure retention point, the controller 230 may use a contact pressure retention control to control the pressurizer 240 to maintain a constant contact pressure by suspending an operation of the pressurizer 240. Under the contact pressure retention control, the pressurizer 240 may not apply or reduce a pressure, but maintain the contact pressure measured at the contact pressure retention point.

In addition, the controller 230 may terminate the measuring of the pressure pulse wave when a preset period of time elapses after the contact pressure retention point. That is, after measuring the amplitude of the pressure pulse wave during a preset period time, the controller 230 may terminate the measuring of the pressure pulse wave by suspending an operation of the sensor 210.

In addition, the pressure applying apparatus 200 may measure a pressure pulse wave from the object 250 when the object 250 is pressed by the pressurizer 240, determine a point in time at which an amplitude of the measured pressure pulse wave changes from increasing to decreasing or from decreasing to increasing, and control the pressurizer 240 to reduce or maintain a pressure applied to press the object 250 after the determined point.

According to example embodiments, a pressure applying apparatus may determine, to be a contact pressure reduction control point for a pressurizer to reduce a contact pressure, a point in time at which an amplitude of a pressure pulse wave changes from increasing to decreasing by continuously applying a pressure at a constant speed, and thus may determine a change pattern of the pressure pulse wave after a maximum amplitude of the pressure pulse wave is measured.

In addition, the pressure applying apparatus may apply a contact pressure retention control to the pressurizer to maintain a contact pressure again based on a change in an amplitude of the pressure pulse wave measured after a contact pressure reduction control for the pressurizer to reduce a contact pressure, thereby expanding a measurement range and achieving a relatively high-frequency resolution in a frequency component analysis of the pressure pulse wave.

Further, the pressure applying apparatus may flexibly control the pressurizer to apply, reduce, or maintain a pressure based on an amplitude of the pressure pulse wave being measured, and thus may obtain various feature values associated with the pressure pulse wave.

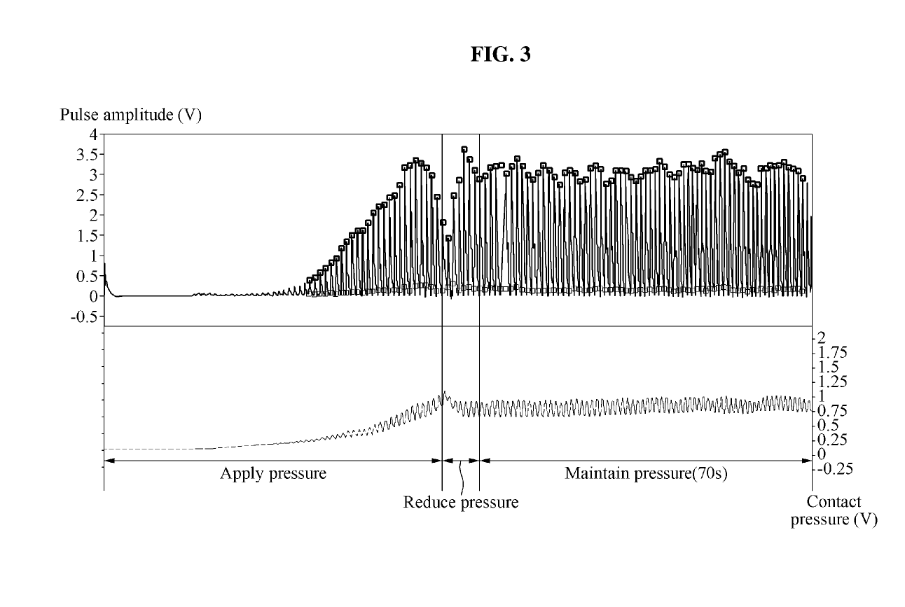

FIG. 3 is a diagram illustrating an example of an amplitude of a pressure pulse wave to be measured by a pressure applying apparatus according to an example embodiment.

As illustrated in FIG. 3, a pressure applying apparatus may continuously apply a pressure to skin above a radial artery, which is an object, at a constant speed using a pressurizer, and measure an amplitude of a pressure pulse wave that gradually increases by the applying of the pressure, for example, as shown in a pressure applying control section in which the pressurizer is controlled to apply a pressure.

In addition, the pressure applying apparatus may detect, as a contact pressure reduction point, a point in time at which the amplitude of the pressure pulse wave being measured changes from increasing to decreasing, for example, as shown in a contact pressure reduction control section in which the pressurizer is controlled to reduce a contact pressure.

Subsequently, the pressure applying apparatus may apply a contact pressure reduced by the pressurizer to the radial artery and sufficiently increase the amplitude of the pressure pulse wave being measured to a previous amplitude, and then continue the measuring of the amplitude of the pressure pulse wave at a fixed contact pressure for a preset period of time and terminate the measuring, as shown in a contact pressure retention control section in which the pressurizer is controlled to maintain a contact pressure.

Here, the pressure applying apparatus may obtain data for an analysis of a time-series feature value from the pressure applying control section, and obtain data for an analysis of a pulse wave and frequency feature value from the contact pressure retention control section.

That is, the pressure applying apparatus may flexibly control the pressurizer configured to apply a pressure to a radial artery in a wrist to add, reduce, or maintain a contact pressure, and thus obtain various pieces of data, for example, a pulse waveform and a contact pressure waveform of the radial artery.

The pulse waveform refers to a waveform of the radial artery to be measured by the pressure applying apparatus, and the contact pressure waveform refers to a degree of an applied pressure or contact pressure to be measured by the pressure applying apparatus. The contact pressure waveform is indicated as a form from which an alternating current (AC) component is actually removed from the pulse waveform. That is, the AC component in the data obtained through the measuring may be the pulse waveform, and a direct current (DC) component in the data may be the contact pressure waveform.

The pressure applying control section refers to a section in which the pressurizer slowly presses a blood vessel of the radial artery, and a pulse wave of the radial artery may increase gradually. In the pressure applying control section, the pulse wave of the radial artery may increase gradually, and then decrease when a maximum amplitude of the pulse wave is obtained and then a pressure is applied further. The pulse waveform may disappear when a pressure is applied to such an extent that the blood vessel is blocked.

The contact pressure reduction control section refers to a section in which the pressurizer is controlled to reduce a contact pressure at a point in time at which an amplitude of the pulse wave of the radial artery is reversed to decreasing after a maximum amplitude of the pulse wave of the radial artery is measured. In the contact pressure reduction control section, the pulse wave of the radial artery may be reversed from decreasing to increasing again.

The contact pressure retention control section refers to a section in which the pressurizer is controlled to detect a point in time at which the reversed amplitude of the pulse wave of the radial artery has a maximum value again, and maintain a contact pressure measured at the detected point until after the point.

In another example, the pressure applying apparatus may flexibly control a contact pressure to increase or decrease based on whether a pressure pulse wave is measured.

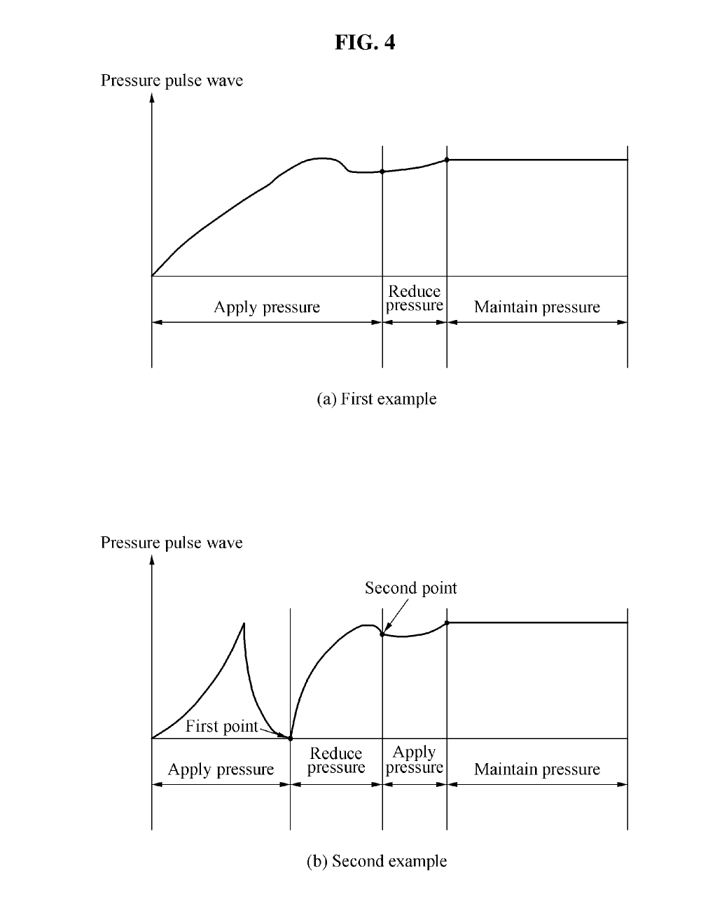

FIG. 4 is a diagram illustrating examples of a method of controlling a contact pressure by a pressure applying apparatus according to an example embodiment.

An (a) portion of FIG. 4 is a diagram illustrating a pressure pulse wave measured according to an example embodiment of applying, reducing, and maintaining a contact pressure, hereinafter referred to as a first example embodiment described with reference to FIG. 3. A (b) portion of FIG. 4 is a diagram illustrating a pressure pulse wave measured according to an example embodiment of applying, reducing, applying, and maintaining a contact pressure, hereinafter referred to as a second example embodiment.

According to the second example embodiment, as illustrated in the (b) portion of FIG. 4, a pressure applying apparatus may continuously measure a pressure pulse wave from an object, or a target from which the pressure pulse wave is to be measured.

The pressure applying apparatus controls a pressurizer to continuously apply a pressure to the object in a pressure applying section. When the pressure is applied, a pressure pulse wave being measured may increase gradually and reach a maximum amplitude, and then rapidly decrease to be 0 when the pressure is applied to such an extent that a blood vessel is blocked. According to the second example embodiment, a point in time at which the pressure pulse wave is not measured is set to be a first point.

The pressure applying apparatus controls the pressurizer to reduce the pressure applied to the object in a contact pressure reduction section after the first point. When the contact pressure is reduced, the amplitude of the pressure pulse wave being measured may increase and then be reversed to decrease at a certain point. According to the second example embodiment, a point in time at which the amplitude of the pressure pulse wave starts decreasing from increasing by reducing the contact pressure is set to be a second point.

The pressure pulse wave to be measured in the contact pressure reduction section is shown in a parabolic form that is upwards in a right side as illustrated in the (b) portion of FIG. 4. In contrast, the pressure pulse wave to be measured in the contact pressure reduction section according to the first example embodiment is shown in an inverse parabolic form that is downwards in a right side as illustrated in the (a) portion of FIG. 4. That is, the pressure pulse wave to be measured in the contact pressure reduction section according to the second example embodiment and the pressure pulse wave to be measured in the contact pressure reduction section according to the first example embodiment may be symmetrical to each other.

The pressure applying apparatus may control the pressurizer to apply a pressure again to the object in a pressure applying section after the second point. Here, the pressure applying apparatus may control the pressurizer more finely to maintain a pressure to be applied again to be the same as the amplitude of the pressure pulse wave measured at the second point. When a preset period of time elapses after the pressure is applied again and then maintained, the pressure applying apparatus may terminate the measuring of the pressure pulse wave.

According to the second example embodiment, by controlling a contact pressure in various ways, the pressure applying apparatus may flexibly measure a pressure pulse wave based on an environment and condition under which the pressure pulse wave is measured.

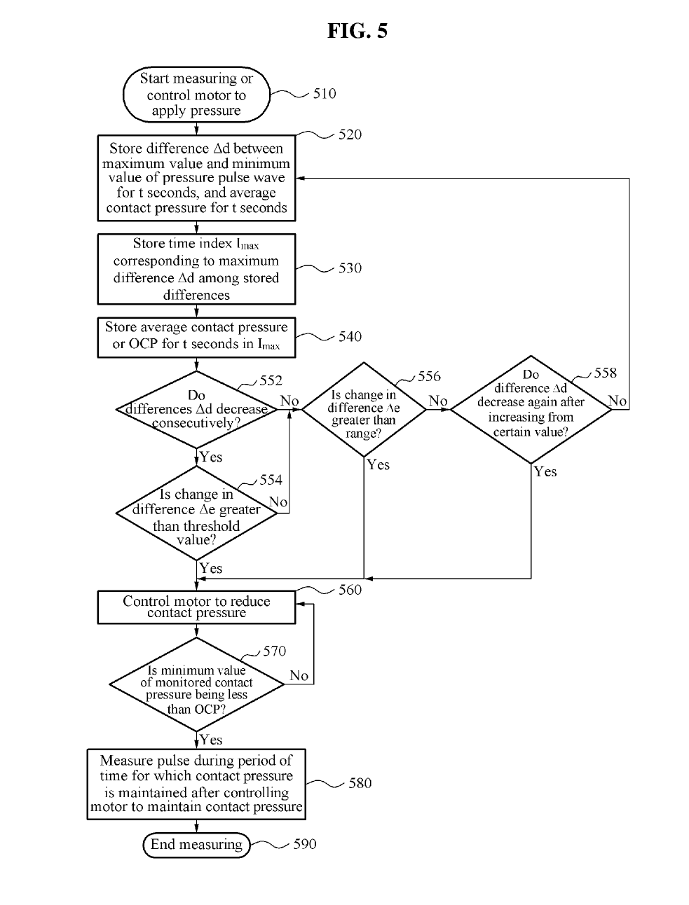

FIG. 5 is a flowchart illustrating a method of controlling a pressurizer by determining a contact pressure reduction point and a contact pressure retention point according to an example embodiment.

Referring to FIG. 5, in operation 510, a pressure applying apparatus starts measuring a pressure pulse wave by controlling a pressurizer to apply a pressure to an object. The pressurizer may be controlled to apply the pressure to the object, and the pressure applying apparatus may measure the pressure pulse wave from the object.

In operation 520, the pressure applying apparatus calculates a difference Δd between a maximum measured value and a minimum measured value of the pressure pulse wave for t seconds, and stores the calculated difference Δd. Here, t indicates 2 seconds, for example. The difference Δd refers to a difference between a maximum value of the pressure pulse wave, or a pulse waveform, measured for t seconds, or 2 seconds, and a minimum value of the pressure pulse wave measured for t seconds. During the measuring, the pressure applying apparatus may calculate the difference Δd every t seconds, or 2 seconds. For example, the pressure applying apparatus may store, in a memory in real time, a contact pressure of the pressure pulse wave in a fourth channel. Simultaneously, the pressure applying apparatus may calculate an average contact pressure for t seconds, or 2 seconds, and store the calculated average contact pressure.

In operation 530, the pressure applying apparatus stores a time index Iₘₐₓ corresponding to a maximum difference Δd among the stored differences Δd. For example, the pressure applying apparatus may store the time index Iₘₐₓ corresponding to a point in time at which the maximum difference Δd appears among the differences Δd that are continuously stored until a current point.

In operation 540, the pressure applying apparatus stores an average contact pressure for t seconds in the stored time index Iₘₐₓ. The pressure applying apparatus may select the average contact pressure for t seconds in the stored time index Iₘₐₓ to be an OCP at which a maximum amplitude of the pressure pulse wave is to be measured.

In operations 552 through 558, the pressure applying apparatus determines whether a condition for controlling the pressurizer to reduce a contact pressure is satisfied.

In detail, in operation 552, the pressure applying apparatus determines whether the differences Δd decrease consecutively. For example, the pressure applying apparatus verifies whether the differences Δd decrease consecutively for 6 seconds. In addition, the pressure applying apparatus verifies whether a difference Δd 2 seconds ago is greater than a difference Δd 4 seconds ago by 0.15 v or higher.

In operation 554, when the condition in operation 552 is satisfied, the pressure applying apparatus determines whether a difference Ae between the consecutively decreased differences Δd is greater than a preset threshold value. For example, when a difference Δd 6 seconds ago is 0.6 v and the difference Δd 2 seconds ago is 0.1 v, the pressure applying apparatus may determine that a difference Ae between the difference Δd 6 seconds ago and the difference Δd 2 seconds ago is 0.5 v and verify whether the difference Ae is greater than or equal to the threshold value, for example, 0.4 v. Here, in the presence of a difference Δd greater than or equal to 2.5 v among all the differences Δd, the pressure applying apparatus may set the threshold value to be 0.5 v. Conversely, in the absence of such a difference Δd greater than or equal to 2.5 v, the pressure applying apparatus may set the threshold value to be 0.4 v.

When a condition in operation 554 is satisfied, the pressure applying apparatus determines that the condition for controlling the pressurizer to reduce the contact pressure is satisfied.

In operation 556, when the condition in operation 552 or 554 is not satisfied, the pressure applying apparatus determines that the difference Ae between the differences Δd changes greatly than a preset range. That is, the pressure applying apparatus may verify whether a difference Ae between the difference Δd 4 seconds ago and the difference Δd 2 seconds ago changes rapidly, for example, by 1.2 v or higher. In addition, in a case in which a blood vessel is blocked and then an amplitude of a pressure pulse wave decreases, or in a case in which the pressurizer presses hard against a bone and an amplitude of a pressure pulse wave increases rapidly, the pressure applying apparatus may verify a rapid change in the difference Ae between the differences Δd.

When a condition in operation 556 is satisfied, the pressure applying apparatus determines that the condition for controlling the pressurizer to reduce the contact pressure is satisfied.

In operation 558, when the condition in operation 556 is not satisfied, the pressure applying apparatus determines whether the difference Δd increases continuously to a preset value or greater, and then decreases subsequently. For example, the pressure applying apparatus may determine whether the difference Δd decreases again to such an extent that a blood vessel is closed. When the difference Δd increases to a preset value, for example, 1.8 v, or greater, the pressure applying apparatus may determine that a sufficient pulse is obtained. When the difference Δd decreases again to a preset value, for example, 1.5 v, or less during continuous monitoring, the pressure applying apparatus may determine that a blood vessel is slowly closed.

When a condition in operation 558 is satisfied, the pressure applying apparatus determines that the condition for controlling the pressurizer to reduce the contact pressure is satisfied.

When a condition in any one of operations 552 through 558 is not satisfied, the pressure applying apparatus returns to operation 520 and repeats subsequent operations.

In operation 560, when the condition for controlling the pressurizer to reduce the contact pressure is satisfied, the pressure applying apparatus controls the pressurizer to reduce the contact pressure. Based on the controlling, the pressurizer reduces the contact pressure.

In operation 570, the pressure applying apparatus determines whether a minimum value of a monitored contact pressure is less than the OCP. For example, the pressure applying apparatus may verify whether a minimum contact pressure monitored in the fourth channel by controlling the pressurizer to reduce the contact pressure becomes less than the stored OCP.

In operation 580, when the contact pressure measured during the pressurizer being controlled to reduce the contact pressure is verified to be less than the OCP, the pressure applying apparatus suspends an operation of the pressurizer and measures a pulse during a period of time for which the contact pressure is maintained. Here, the suspending of the operation of the pressurizer indicates maintaining a current state without applying or reducing a contact pressure by the pressurizer. That is, after the pressurizer is controlled to apply a pressure, the pressure applying apparatus may compare a contact pressure of the pressurizer being controlled to reduce the contact pressure to the OCP, which is a contact pressure at a point in time at which a maximum pressure pulse wave is obtained. Here, when a minimum contact pressure is less than the OCP, the pressure applying apparatus may suspend the operation of the pressurizer to maintain the contact pressure to be the OCP. Subsequently, the pressure applying apparatus may measure a pressure pulse wave while maintaining the contact pressure during a preset contact pressure retention time.

In operation 590, when the contact pressure retention time elapses, the pressure applying apparatus terminates the measuring.

Hereinafter, a flow of operations of the pressure applying apparatus 200 will be described in detail.

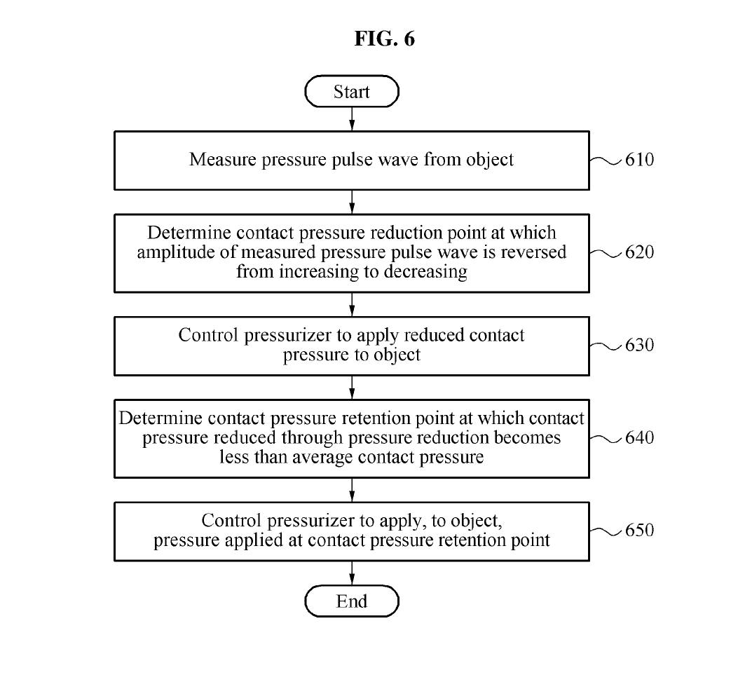

FIG. 6 is a flowchart illustrating a pressure applying method according to an example embodiment.

The pressure applying method to be described hereinafter may be performed by a pressure applying apparatus described above.

In operation 610, the pressure applying apparatus measures a pressure pulse wave from an object. In operation 610, a blood vessel of the object may be gradually contracted by a pressure applied by a pressurizer and a pulse wave may be generated, and thus the pressure applying apparatus may measure an amplitude of the pressure pulse wave based on the generated pulse wave. The amplitude of the pressure pulse wave to be measured may be measured by a unit of v.

In operation 610, the pressure applying apparatus calculates an OCP associated with the applying of the pressure. The OCP refers to a contact pressure measured at a point in time at which a maximum amplitude of the pressure pulse wave occurs.

To calculate the OCP, the pressure applying apparatus may calculate a difference between a maximum measured value and a minimum measured value of the amplitude of the pressure pulse wave in each preset time interval during the pressurizer applying the pressure to the object. Subsequently, the pressure applying apparatus may calculate, as the OCP, an average contact pressure in a time interval having a maximum difference among the calculated differences. Here, the average contact pressure refers to a mean value of contact pressures measured during a certain period of time.

For example, the pressure applying apparatus may calculate the difference Δd between the maximum measured value and the minimum measured value of the amplitude of the pressure pulse wave measured at each time interval of 2 seconds during the pressurizer being controlled to apply the pressure to the object, and calculate a mean value of contact pressures measured in a time interval at each time interval of 2 seconds. Subsequently, the pressure applying apparatus may recognize a time interval Iₘₐₓ at which a maximum difference among differences Δd calculated consecutively until a current point, and determine a mean value of contact pressures calculated in the recognized time interval Iₘₐₓ to be the OCP.

In operation 620, the pressure applying apparatus determines a contact pressure reduction point at which the amplitude of the pressure pulse wave measured by applying the pressure to the object through the pressurizer is reversed from increasing to decreasing. In operation 620, the pressure applying apparatus determines a point in time at which the amplitude of the pressure pulse wave is reversed to decreasing from the maximum measured value to be a point in time at which a control method for the pressurizer changes from a pressure applying control to a contact pressure reduction control.

When determining the contact pressure reduction point, the pressure applying apparatus may numerically determine the contact pressure reduction point using the OCP and the difference Δd between the maximum measured value and the minimum measured value of the amplitude of the pressure pulse wave.

In one example (example 1), when a plurality of calculated differences decrease consecutively, the pressure applying apparatus may determine the contact pressure reduction point.

For example, under the assumption that, based on a current point, a difference between a maximum measured value and a minimum measured value of the pressure pulse wave in a previous time interval between 6 seconds and 4 seconds is Δdₙ₋₃, a difference between maximum and minimum values of the pressure pulse wave in a previous time interval between 4 seconds and 2 seconds is Δdₙ₋₂, and a difference between maximum and minimum values of the pressure pulse wave in a previous time interval between 2 seconds and 0 second is Δdₙ₋₁, and the three differences Δdₙ₋₃, Δdₙ₋₂, and Δdₙ₋₁ calculated for 6 seconds decrease consecutively, the pressure applying apparatus may determine the contact pressure reduction point by determining a point in time at which the amplitude of the pressure pulse wave is reversed to decrease from the maximum measured value.

For another example, the pressure applying apparatus may determine the contact pressure reduction point by determining that the differences decrease consecutively when the difference Δdₙ₋₁ is not greater than Δdₙ₋₂ by 0.15 v or higher.

In another example (example 2), when a lastly calculated second difference among the consecutively calculated differences is less than an initially calculated first difference, and a difference between the first difference and the second difference is greater than a preset first threshold value, the pressure applying apparatus may determine the contact pressure reduction point. For example, when the initially calculated first difference is the difference Δdₙ₋₃ among the three differences Δdₙ₋₃, Δdₙ₋₂, and Δdₙ₋₁, and the lastly calculated second difference is the difference Δdₙ₋₁ among the three differences Δdₙ₋₃, Δdₙ₋₂, and Δdₙ₋₁, the pressure applying apparatus may compare the first difference Δdₙ₋₃ and the second difference Δdₙ₋₁. Here, when the second difference Δdₙ₋₁ is less than the first difference Δdₙ₋₃ and a difference Ae between the first difference Δdₙ₋₃ and the second difference Δdₙ₋₁ is greater than the first threshold value, for example, 0.4 v, the pressure applying apparatus may determine the contact pressure reduction point by determining a point in time at which the amplitude of the pressure pulse wave is reversed from the maximum measured value.

Here, when any one of differences Δd monitored continuously is greater than or equal to 2.5 v, the pressure applying apparatus may adjust the first threshold value to be 0.5 v.

Example 2 described in the foregoing may be implemented independently, or implemented depending on a condition that example 1 is satisfied.

In still another example (example 3), when, among the consecutive differences, a difference between two adjacent calculated differences is greater than a preset second threshold value, the pressure applying apparatus may determine the contact pressure reduction point.

For example, in a case in which, among the three differences Δdₙ₋₃, Δdₙ₋₂, and Δdₙ₋₁, a difference Ae between two adjacent calculated differences Δdₙ₋₂ and Δdₙ₋₁ exceeds the second threshold value, for example, 1.2 v, the pressure applying apparatus may determine the contact pressure reduction point by determining a point in time at which the amplitude of the pressure pulse wave is reversed from the maximum measured value.

That is, in example 3 described in the foregoing, the pressure applying apparatus may determine the contact pressure reduction point by verifying whether the amplitude of the pressure pulse wave decreases by a suddenly blocked blood vessel or rapidly increases by the pressurizer being pressed hard against a bone.

Example 3 described in the foregoing may be implemented independently, or implemented depending on a condition that both examples 1 and 2 are not satisfied.

In yet another example (example 4), when a difference above a preset threshold range is calculated and then a difference below the threshold range is calculated, the pressure applying apparatus may determine the contact pressure reduction point.

For example, under the assumption that the threshold range is 1.8 v to 1.5 v and, among the three differences Δdₙ₋₃, Δdₙ₋₂, and Δdₙ₋₁, the difference Δdₙ₋₃ is calculated to be greater than an uppermost value 1.8 v of the threshold range and then one of the differences Δdₙ₋₂ and Δdₙ₋₁ is calculated to be less than a lowermost value 1.5 v of the threshold range, the pressure applying apparatus may determine the contact pressure reduction point by determining a point in time at which the amplitude of the pressure pulse wave is reversed from the maximum measured value.

In example 4 described in the foregoing, the pressure applying apparatus may determine the contact pressure reduction point by determining that a sufficient pulse is generated when a difference Δd is above the threshold range, and that another difference Δd is out of the threshold range through continuous monitoring and a blood vessel is slowly closed.

Example 4 described in the foregoing may be implemented independently, or implemented depending on a condition that examples 1 through 3 are not satisfied.

In operation 630, the pressure applying apparatus controls the pressurizer to reduce the pressure applied to the object after the contact pressure reduction point. In operation 630, based on the determination of the contact pressure reduction point, the pressure applying apparatus may change the control method for the pressurizer from the pressure applying control to the contact pressure reduction control.

Thus, by increasing again the amplitude of the pressure pulse wave that is reversed to decreasing from the maximum measured value, continuously verifying a change pattern of the pressure pulse wave after the maximum amplitude of the pressure pulse wave is measured may be enabled.

In operation 640, the pressure applying apparatus continuously monitors a contact pressure by the pressurizer controlled to reduce the contact pressure, and determines a contact pressure retention point at which the contact pressure becomes less than the OCP. In operation 640, the pressure applying apparatus determines the contact pressure retention point using the OCP calculated during the pressurizer being controlled to apply a pressure and a contact pressure measured during the pressurizer being controlled to reduce the contact pressure.

For example, after the pressurizer is controlled to reduce the contact pressure, the pressure applying apparatus may monitor the contact pressure by the pressurizer and determine, to be the contact pressure retention point, a point in time at which a gradually reduced contact pressure becomes less than the OCP.

In operation 650, the pressure applying apparatus controls the pressurizer to maintain a contact pressure measured at the contact pressure retention point until after the contact pressure retention point. In operation 650, based on the determination of the contact pressure retention point, the pressure applying apparatus may suspend an operation of the pressurizer to control the pressurizer to maintain a constant pressure to be applied to the object. The pressurizer that is controlled to maintain the contact pressure may continuously maintain the contact pressure measured at the contact pressure retention point without applying or reducing a contact pressure.

When a preset period of time elapses after the contact pressure retention point, the pressure applying apparatus may terminate the measuring of the pressure pulse wave. That is, the pressure applying apparatus may terminate the measuring of the pressure pulse wave by suspending an operation of a sensor after measuring the amplitude of the pressure pulse wave during a preset contact pressure retention time.

According to an example embodiment, a pressure applying method may include measuring a pressure pulse wave from an object when the object is pressed by a pressurizer, determining a point in time at which an amplitude of the measured pressed pulse wave changes from increasing to decreasing or from decreasing to increasing, and controlling the pressurizer to reduce or maintain a pressure applied to press the object after the determined point.

According to an example embodiment, a pressure applying method may include measuring a pressure pulse wave from an object, determining a second point at which an amplitude of the pressure pulse wave is reversed from increasing to decreasing by applying a pressure to the object until a first point at which the pressure pulse wave is not measured and then reducing a contact pressure after the first point, and controlling a pressurizer to apply a pressure to the object after the second point.

Thus, in the pressure applying method, by recognizing, as a contact pressure reduction control point for the pressurizer to reduce a contact pressure, a point in time at which an amplitude of a pressure pulse wave changes from increasing to decreasing by continuously applying a pressure at a constant speed, verifying a change pattern of the pressure pulse wave after a maximum amplitude of the pressure pulse wave is measured may be enabled.

In addition, in the pressure applying method, by controlling the pressurizer to maintain the contact pressure again based on a change in the amplitude of the pressure pulse wave after the pressurizer is controlled to reduce the contact pressure, a measurement range may be expanded, and also a relatively high-frequency resolution may be obtained in a frequency component analysis of the pressure pulse wave.

Further, in the pressure applying method, by flexibly controlling the pressurizer to apply, reduce, or maintain a contact pressure based on the amplitude of the pressure pulse wave, a wide range of various feature values associated with the pressure pulse wave may be obtained.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher level code that may be executed by the computer using an interpreter. The above-described devices may be configured to act as one or more software modules in order to perform the operations of the above-described example embodiments, or vice versa.

A number of example embodiments have been described above. Nevertheless, it should be understood that various modifications may be made to these example embodiments. For example, suitable results may be achieved if the described techniques are performed in a different order and/or if components in a described system, architecture, device, or circuit are combined in a different manner and/or replaced or supplemented by other components or their equivalents.

Accordingly, other implementations are within the scope of the following claims.

1. A pressure applying method comprising:
measuring a pressure pulse wave from an object;
determining a contact pressure reduction point at which an amplitude of the measured pressure pulse wave is reversed from increasing to decreasing by applying a pressure to the object through a pressurizer; and
controlling the pressurizer to reduce the pressure applied to the object after the contact pressure reduction point.

2. The pressure applying method of claim 1, further comprising:
calculating an optimal contact pressure (OCP) associated with the applying of the pressure;
determining a contact pressure retention point at which a contact pressure reduced through the reducing of the pressure becomes less than the OCP; and
controlling the pressurizer to maintain a contact pressure measured at the contact pressure retention point until after the contact pressure retention point.

3. The pressure applying method of claim 2, further comprising:
terminating the measuring of the pressure pulse wave when a preset period of time elapses after the contact pressure retention point.

4. The pressure applying method of claim 2, further comprising:
calculating a difference between a maximum measured value and a minimum measured value of the amplitude of the pressure pulse wave in each preset time interval prior to the contact pressure reduction point,
wherein the calculating of the OCP comprises:
calculating, as the OCP, an average contact pressure in a time interval having a maximum value among the calculated differences.

5. The pressure applying method of claim 1, further comprising:
calculating a difference between a maximum measured value and a minimum measured value of the amplitude of the pressure pulse wave in each preset time interval prior to the contact pressure reduction point,
wherein the determining of the pressure reduction point comprises:
determining the contact pressure reduction point when a plurality of calculated differences decrease consecutively; or
determining the contact pressure reduction point when a lastly calculated second difference among the calculated differences is less than an initially calculated first difference among the differences, and a difference between the first difference and the second difference is greater than a preset first threshold value.

6. The pressure applying method of claim 5, wherein the determining of the contact pressure reduction point further comprises:
determining the contact pressure reduction point when a difference between two adjacent calculated differences among the calculated differences is greater than a preset second threshold value.

7. The pressure applying method of claim 6, wherein the determining of the contact pressure reduction point further comprises:
determining the contact pressure reduction point when a difference below a preset threshold range is calculated after a difference above the threshold range is calculated.

8. A pressure applying method comprising:
measuring a pressure pulse wave from an object by pressing the object by a pressurizer;
determining a point in time at which an amplitude of the measured pressure pulse wave changes from increasing to decreasing or from decreasing to increasing; and
controlling the pressurizer to reduce or maintain a pressure applied to press the object after the determined point.

9. A pressure applying method comprising:
measuring a pressure pulse wave from an object;
determining a second point at which an amplitude of the pressure pulse wave is reversed from increasing to decreasing by applying a pressure to the object until a first point at which the pressure pulse wave is not measured and reducing the pressure after the first point; and
controlling the pressurizer to apply a pressure to the object after the second point.

10. A pressure applying apparatus comprising:
a sensor configured to measure a pressure pulse wave from an object;
a processor configured to determine a contact pressure reduction point at which an amplitude of the pressure pulse wave measured by applying a pressure to the object through a pressurizer is reversed from increasing to decreasing; and
a controller configured to control the pressurizer to reduce the pressure applied to the object after the contact pressure reduction point.

11. The pressure applying apparatus of claim 10, wherein, when the sensor calculates an optimal contact pressure (OCP) associated with the applying of the pressure, the processor is configured to determine a contact pressure retention point at which a contact pressure reduced through the reducing of the pressure becomes less than the OCP, and
the controller is configured to control the pressurizer to maintain a contact pressure measured at the contact pressure retention point until after the contact pressure retention point.

12. The pressure applying apparatus of claim 11, wherein the controller is configured to control the sensor to terminate the measuring of the pressure pulse wave when a preset period of time elapses after the contact pressure retention point.

13. The pressure applying apparatus of claim 11, wherein the sensor is configured to calculate a difference between a maximum measured value and a minimum measured value of the amplitude of the pressure pulse wave in each preset time interval prior to the contact pressure reduction point, and
the sensor is configured to calculate, as the OCP, an average contact pressure in a time interval having a maximum value among the calculated differences.

14. The pressure applying apparatus of claim 10, wherein the sensor is configured to calculate a difference between a maximum measured value and a minimum measured value of the amplitude of the pressure pulse wave in each present time interval prior to the contact pressure reduction point, and
the processor is configured to determine the contact pressure reduction point when a plurality of calculated differences decrease consecutively, or when a lastly calculated second difference among the calculated differences is less than an initially calculated first difference and a difference between the first difference and the second difference is greater than a preset first threshold value.

15. The pressure applying apparatus of claim 14, wherein, when a difference between two adjacent calculated differences among the calculated differences is greater than a preset second threshold value, the processor is configured to determine the contact pressure reduction point.

16. The pressure applying apparatus of claim 15, wherein, when a difference below a preset threshold range is calculated after a difference above the threshold range is calculated, the processor is configured to determine the contact pressure reduction point.

17. A pressure applying apparatus configured to measure a pressure pulse wave from an object by pressing the object by a pressurizer, determine a point in time at which an amplitude of the measured pressure pulse wave changes from increasing to decreasing or from decreasing to increasing, and control the pressurizer to reduce or maintain a pressure applied to press the object after the determined point.

18. A pressure applying apparatus configured to measure a pressure pulse wave from an object, determine a second point in time at which an amplitude of the pressure pulse wave is reversed from increasing to decreasing by applying a pressure to the object until a first point in time at which the pressure pulse wave is not measured and reducing the pressure after the first point, and control the pressurizer to apply the pressure to the object after the second point.
